(19) United States
(12) Patent Application Publication
(10) Pub. No.: US 2024/0016704 A1
(43) Pub. Date: Jan. 18, 2024

(54) DENTAL PRODUCT FOR FORMING AN ENDODONTIC CEMENT

(71) Applicant: PRODUITS DENTAIRES PIERRE ROLLAND, Merignac (FR)

(72) Inventors: Vincent MAURAT, Pessac (FR); Marine LANDRODIE, Riberac (FR)

(21) Appl. No.: 18/039,798

(22) PCT Filed: Nov. 15, 2021
(86) PCT No.: PCT/FR2021/052017

§ 371 Date: Jun. 1, 2023

(30) Foreign Application Priority Data
Dec. 2, 2020 (FR) FR2012532

(51) Int. Cl. A61K 6/54; A61K 6/838; A61K 6/17; A61K 6/73

(52) U.S. Cl. CPC A61K 6/54; A61K 6/838; A61K 6/17; A61K 6/73

(57) ABSTRACT

A dental product is provided for forming a hydroxyapatite-based endodontic cement in the presence of moisture. The formed cement is suitable for simple ultrasonic tip reworking.

### TECHNICAL FIELD

The invention relates to a dental product for forming an endodontic cement, or root canal cement, in the presence of moisture in a dental canal. The cement formed is able to be easily reworked, for example by applying ultrasound.

### PRIOR ART

Endodontic treatment is a tricky procedure that underpins periodontal health and the durability of the dental organ. It consists in preventing, diagnosing and treating dental pulp and periapical infections (in the bone and around the roots) in order to transform a pathological tooth into a healthy, asymptomatic and functional entity on the arch.

Root canal or endodontic treatment is indicated for irreversible pulpitis (inflammation, pulp infection) or necrotic pulp with or without clinical and/or radiographic signs of apical periodontitis (inflammatory lesions of the periodontium) or for living pulp if the prognosis for pulp vitality is unfavorable, if there is a root amputation or hemisection and if there is a high probability of pulp exposure during the coronal restoration not allowing direct capping.

Inflammation and infection of the pulp can be caused by many things, such as caries, cracks, fractures or tooth impact. If the infected or inflamed pulp is left untreated, it can cause severe pain and even lead to an abscess.

Endodontic treatment consists in preventing or banishing infection by removing bacteria and their toxins from the root canal system as well as all residues that may serve as nutrients and support for bacterial multiplication. In order to maintain this disinfection, the pulp or its remnants in a healthy root canal system must be replaced with a watertight filling that promotes healing. This procedure eliminates and neutralizes organic and bacterial substances that cause irritation to the periapical tissues. The result must be stable and durable. Once restored, the tooth must be functional, asymptomatic and show no clinical signs.

Root canal cement is known to be well tolerated and have good durability. However, reworking of the previously placed dental cement in the event of the need to repeat endodontic treatment, for example to restore the cement, can be tricky. Indeed, it may be necessary to use potentially toxic solvents to reduce the hardness of the cement in the canal for the reworking procedure.

It is desirable to have dental products available for forming an endodontic cement that will produce the desired root canal filling while making a reworking procedure easier.

### DISCLOSURE OF THE INVENTION

The invention relates to a dental product for forming an endodontic cement in a dental canal, said product comprising at least:
a powder composition in a mass content comprised between 40% and 55% comprising at least one calcium phosphate and able to form hydroxyapatite in the presence of moisture,
a binder in a mass content comprised between 0.5% and 5%, and
a liquid medium in a mass content comprised between 30% and 50%.

The particular composition of the dental product described above confers several advantages on the endodontic cement obtained.

Indeed, this cement is biocompatible and non-irritating. It is based on hydroxyapatite, which is the main constituent of the dentine, and it sets naturally when the product is brought into contact with the moisture in the canal. The cement obtained also has sufficient cohesion to provide the desired strength without this being too great for an easy and safe reworking procedure, for example with an ultrasonic tip. Thus, the cement advantageously has a compressive strength comprised between 3 MPa and 15 MPa, for example comprised between 3 MPa and 10 MPa. The viscosity of the product is further limited so that it can be easily introduced into the dental canal without forming air bubbles. Thus, the dynamic viscosity of the product is advantageously less than or equal to 240 Pa·s, for example less than or equal to 200 Pa·s, for example less than or equal to 150 Pa·s. The viscosity can be determined by a planar rheometer, for example marketed under the name “Discovery HR-2” by TA instruments, at A “flow ramp” method of 0.01 to 10 s⁻¹ and a “flow sweep” method of 10 to 1·10⁻⁶ s⁻¹ can be used to measure the viscosity and the flow threshold of the dental product.

More precisely, the content of powder composition is sufficient for the compressive strength of the cement to be sufficient to obtain the desired cohesion while remaining limited to allow the reworking procedure and for the product to retain a controlled viscosity. The liquid medium, present in the product in a significant content, also helps limit the viscosity. The binder content is sufficient to help obtain the desired cohesion while remaining limited so as not to affect the viscosity.

It will be noted that the dental product having the composition described above corresponds to the product ready to be introduced into the dental canal for forming cement. The product may be packaged as a single composition comprising all of its constituents prior to use or in several separate parts, each in a separate compartment, to be mixed only at the time of use of the product, as will be described in greater detail below. Unless otherwise stated, the contents of the constituents of the dental product are taken before introduction into the dental canal.

In an example embodiment, the mass content of the powder composition is comprised between 43% and 52%.

Such a feature advantageously helps optimize the compromise between the compressive strength of the cement and the viscosity of the product.

In an example embodiment, the powder composition comprises a mixture of dicalcium phosphate and tetracalcium phosphate.

In particular, the ratio between the mass of tetracalcium phosphate in the product and the mass of dicalcium phosphate in the product may be comprised between 0.9 and 1.9.

In an example embodiment, the powder composition has an average particle size D50 of less than or equal to 50 μm.

“Average size D50” refers to the size given by the statistical particle size distribution to half of the population.

The choice of fine particles for the powder composition helps to achieve improved cohesion and rapid setting of the cement in the dental canal.

In an example embodiment, the mass content of the binder is comprised between 0.5% and 3.5%.

Such a feature advantageously helps optimize the compromise between the compressive strength of the cement and the viscosity of the product.

In an example embodiment, the product further comprises a radiopaque contrast agent in a mass content of less than or equal to 15%.

The presence of the radiopaque contrast agent allows the monitoring and radiographic inspection of the cement during its placement. When it is present, its content must nevertheless remain limited so as not to alter the compressive strength of the cement by affecting the formation of the network by the calcium phosphates.

In an example embodiment, the product further comprises a suspending agent in a mass content of less than or equal to 1.5%.

The presence of the suspending agent avoids any risk of separation between the solid and liquid phases in the product. When it is present, its content must nevertheless remain limited so as not to excessively increase the viscosity of the product.

In an example embodiment, the product further comprises a cement setting accelerator in a mass content of less than or equal to 10%.

This compound accelerates the formation of cement. When present, its content must nevertheless remain limited so as not to excessively increase the viscosity of the product.

In an example embodiment, the product is such that:
the mass content of powder composition is comprised between 43% and 52%,
the mass content of binder is comprised between 0.5% and 1%,
the liquid medium is non-aqueous and its mass content is comprised between 30% and 40%,
the product comprises a radiopaque contrast agent in a mass content comprised between 5% and 15%, for example between 5% and 12%, and
the product comprises a cement setting accelerator in a mass content comprised between 4% and 7%.

In particular, the radiopaque contrast agent may comprise barium sulfate and the mass content of the powder composition may be comprised between 43% and 48%.

Alternatively, the radiopaque contrast agent may comprise zirconium oxide and the mass content of the powder composition may be comprised between 48% and 52%.

According to another example embodiment, the product is such that:
the mass content of powder composition is comprised between 43% and 52%,
the mass content of binder is comprised between 3% and 3.5%,
the liquid medium comprises water and its mass content is comprised between 30% and 40%, and
the product comprises a radiopaque contrast agent in a mass content comprised between 5% and 15%.

The invention also relates to a kit for filling and sealing a dental canal, comprising at least the product as described above in an introduction device suitable for introducing the product into the dental canal.

The invention also relates to a process for placing an endodontic cement comprising at least:
introducing a dental product as described above into a dental canal, and
setting the dental product thus introduced in contact with the moisture present in the dental canal to form the endodontic cement.

The invention also relates to a process for treating an endodontic cement obtained from the dental product as described above and present in a dental canal wherein said endodontic cement is brought into contact with a tool vibrating at an ultrasonic frequency.

### BRIEF DESCRIPTION OF THE DRAWINGS

FIG. 1 provides photographs of specimens and tooth models filled with different root canal cements.

FIG. 2 provides photographs of tooth models after root canal cement has been retreated.

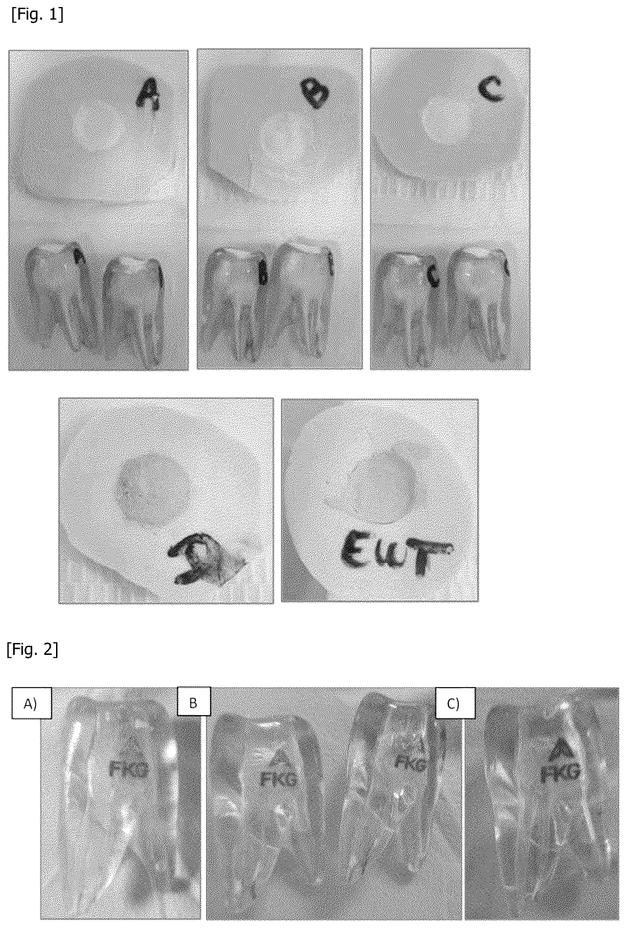

### DESCRIPTION OF THE EMBODIMENTS

The dental product is in fluid form and comprises at least the powder composition and the binder in the liquid medium. The dental product may be in the form of a suspension. The dental product is intended to be introduced into a dental canal to form an endodontic cement for filling and sealing this canal. The dynamic viscosity of the dental product may be comprised between Pa·s and 240 Pa·s, for example between 90 Pa·s and 200 Pa·s, or even between 90 Pa·s and 150 Pa·s. As noted above, the compressive strength of the resulting cement may be comprised between 3 MPa and 15 MPa, for example comprised between 3 MPa and 10 MPa.

The compressive strength of the cement can be measured using the operative protocol described below. A plaster of Paris mold with 6 mm diameter and 10 mm height, allowing for water exchange was filled with the dental product using a spatula, taking care not to leave any air bubbles. The filled mold was placed in an oven at 40° C. and high relative humidity for 3 days. After 3 days, the endodontic cement specimen was removed from the mold, taking care not to damage it by subjecting it to excessive forces. The specimen was then subjected to a compressive strength test using the universal testing machine marketed under the name AGS-X by SHIMADZU equipped with the 500 N or 10 kN load cell, the jig holder and the P/25 jig. A test speed of 0.750 mm/minute was imposed during the compression test until the specimen broke. The compressive strength Rc is then determined using the following formula where Fmax is the force applied just prior to specimen failure and d is the initial specimen diameter.

Rc=Fmax/((πxd²)/4) [Math. 1]

In the following, more details on the different components of the dental product are provided. The different features described below can be taken in isolation or in combination with each other.

The dental product comprises a powder composition which is able to form hydroxyapatite Ca₁₀(PO₄)₆(OH)₂ in the presence of moisture, in particular on contact with moisture present in the dental canal into which the product is introduced, in order to form the dental cement.

The powder composition comprises at least one calcium phosphate and may, according to an example, comprise a mixture of an acidic calcium phosphate and a basic calcium phosphate which are able to react in the presence of moisture to form hydroxyapatite. In this case, the acidic and basic calcium phosphates are each in the form of a powder.

Forming hydroxyapatite from at least one calcium phosphate is known per se. Mention may be made of the use of a mixture of dicalcium phosphate, which may be anhydrous of the formula CaHPO₄ or dihydrate of the formula CaHPO₄, 2 H₂O, and tetracalcium phosphate of the formula Ca₄(PO₄)₂O. Dicalcium phosphate is an acidic calcium phosphate and tetracalcium phosphate a basic calcium phosphate. It is also possible to use a powder composition comprising a mixture of a calcium phosphate, such as dicalcium phosphate dihydrate, with calcium carbonate or calcium hydroxide. Nevertheless, in the context of the invention, preferential use will be made of a powder composition comprising, for example consisting essentially of, a mixture of an acidic calcium phosphate, such as dicalcium phosphate, and a basic calcium phosphate, such as tetracalcium phosphate. For example, use may be made of an equimolar mixture of dicalcium phosphate and tetracalcium phosphate which react in the presence of moisture to form hydroxyapatite. The powder composition, according to an example, may be free of calcium carbonate, calcium hydroxide or tricalcium silicate.

The average particle size D50 of the powder composition may be less than or equal to 50 μm, as indicated above. According to an example, when a mixture of acidic calcium phosphate and basic calcium phosphate is used, the average particle size D50 of the basic calcium phosphate may be less than or equal to 10 μm. Such a feature advantageously helps accelerate the setting of the cement. As indicated above, the powder composition is present in the dental product in a mass content comprised between 40% and 55%, for example between 43% and 52%, for example between 45% and 50%. In the case where the powder composition comprises a mixture of dicalcium phosphate and tetracalcium phosphate, the mass ratio [mass of tetracalcium phosphate in the product]/[mass of dicalcium phosphate in the product] may be comprised between 0.9 and 1.9.

The dental product comprises a liquid medium in which the powder composition and binder are present. The liquid medium may comprise at least one of the following compounds: glycerol, propylene glycol, water, and mixtures thereof. The liquid medium may be non-aqueous or, alternatively, comprised of water. It has been indicated above that the dental product may be packaged in a single composition comprising all of its constituents before use. In this case, the use of a non-aqueous liquid medium is preferred in order to avoid the formation of hydroxyapatite prior to use. According to an alternative, the product is packaged in several separate parts, each in a separate compartment, intended to be mixed only at the time of use of the product. In this case, for example, there may be a powder composition comprising a mixture of an acidic calcium phosphate and a basic calcium phosphate and the acidic calcium phosphate is packaged in a first compartment and the basic calcium phosphate is packaged in a second compartment, separate from the first compartment. In this case, water may be used in the first compartment and/or the second compartment, since prior to use the acidic and basic calcium phosphates are separated and are unable to react to form hydroxyapatite. As noted above, the liquid medium is present in the dental product in a mass content comprised between 30% and 50%, for example between 30% and 40%.

The dental product further comprises a binder which is formed from a compound distinct from the compound or compounds forming the powder composition. According to an example, the binder may be selected from: carboxymethyl cellulose, polyvinylpyrrolidone, and mixtures thereof. For example, the product marketed under the name Kollidon® 90 F by BASF may be used. As noted above, the binder is present in the dental product in a mass content comprised between 0.5% and 5%, for example between 0.5% and 3.5%. More particularly in the case where the liquid medium comprises water, the mass content of binder in the dental product may be comprised between 3% and 5%, for example between 3% and 3.5%. Alternatively, when the liquid medium is non-aqueous, the mass content of binder in the dental product may be comprised between 0.5% and 1%.

As noted above, the dental product may comprise other constituents in the liquid medium in addition to the powder composition and the binder. These additional constituents, if present, are described below.

The dental product may further comprise a radiopaque contrast agent which may be selected from: barium sulfate, zirconium oxide, or mixtures thereof. The radiopaque contrast agent may be present in the dental product in a mass content of less than or equal to 15%, for example comprised between 5% and 14%, for example between 5% and 12%.

As noted above, the dental product may further comprise a suspending agent. The suspending agent may comprise silica. For example, the product marketed under the name Aerosil® 200 by Evonik may be used. The suspending agent may be present in the dental product in a mass content of less than or equal to 1.5%, for example less than or equal to 1%. This content may be comprised between 0.25% and 1.5%, for example between 0.25% and 1%, or between 0.5% and 1.5%, for example between 0.5% and 1%.

As noted above, the dental product may further comprise a cement setting accelerator. The setting accelerator may be hydroxyapatite, sodium phosphate, or a mixture thereof. The setting accelerator may be present in the dental product in a mass content of less than or equal to 10%, for example less than or equal to 7% or less than or equal to 6%. This content may be comprised between 4% and 10%, for example between 4% and 7%, for example between 5% and 6%.

The dental product may further comprise at least one preservative. The preservative may be present in the dental product in a mass content of less than or equal to 1.5%, for example less than or equal to 1%. This mass content may be comprised between 0.15% and 1.5%, for example between 0.15% and 1%, or between 0.5% and 1.5%, for example between 0.5% and 1%. The preservative stabilizes the dental product over time and may be selected from: methyl paraben, propyl paraben, chlorhexidine, and mixtures thereof.

The dental product may comprise at least one dental treatment agent, for example a therapeutic agent, such as at least one of an antiseptic or an anti-inflammatory. The antiseptic agent may be selected from chlorhexidine hydrochloride, chlorhexidine dihydrochloride, chlorhexidine gluconate, chlorhexidine digluconate, sodium hypochlorite, quaternary ammoniums, iodine derivatives or mixtures thereof. The antiseptic agent may in particular comprise chlorhexidine gluconate or chlorhexidine digluconate. The anti-inflammatory agent may be selected from butoform, prednisolone acetate, β-glycyrrhetinic acid, a non-steroidal anti-inflammatory or mixtures thereof.

Various details relating to the composition of the dental product have just been described. The following provides details on the use of this product to form the dental cement and to rework it.

The dental product may be packaged as a single composition comprising all of its constituents prior to use, or in several separate parts, each in a separate compartment, intended to be mixed only at the time of use of the dental product, with, for example, separation of the acidic and basic calcium phosphates.

The introduction device for introducing the dental product into the root canal may be a single- or double-compartment syringe with specific tips for filling the canal. Such an introductory device is known per se and, by way of example of an introduction device that can be used, mention may be made of the tool marketed under the name Colibri™ by Sulzer Mixpac™ (double-compartment example). Once introduced into the root canal, the dental product hardens on contact with the moisture in the canal to form hydroxyapatite and thus fill and seal the root canal.

The cement has a moderate hardness allowing it to be easily reworked, for example, to perform a dental cement restoration procedure. The cement may be removed in whole or in part from the canal by contact with a tool vibrating at an ultrasonic frequency, for example an ultrasonic tip. The vibration frequency of the tool may be comprised between 26 kHz and 36 kHz. The tool vibrating at an ultrasonic frequency is known per se and, by way of example of a tool that can be used, mention may be made of the insert No. ET25S marketed by SATELEC.

### EXAMPLES

The table below provides three examples of formulations of dental products according to the invention that have been prepared.

TABLE 1

| | | FORMU- | FORMU- | FORMU- |
|---|---|---|---|---|
| | | LA A | LA B | LA C |
| Liquid medium | Propylene glycol | 36.65% | 32.15% | 17.63% |
| | Water | 0 | 0 | 17.50% |
| Binder | Kollidon 90F | 0.85% | 0.85% | 3.25% |
| Calcium | DCPD | 15.75% | 17.50% | 17.50% |
| phosphates | TTCP | 29.25% | 12.50% | 32.50% |
| | Total calcium | 45.00% | 50.00% | 50.00% |
| | phosphate | | | |
| Suspending agent | Aerosil ® 200 | 0.50% | 1.00% | 0.63% |
| Radiopaque | Barium sulfate/ | 10.00% | 10.00% | 10.00% |
| contrast agent | Zirconium oxide | | | |
| Antiseptic agent | Chlorhexidine | 1.00% | 1.00% | 1.00% |
| Setting | Hydroxyapatite | 6.00% | 5.00% | 0 |
| accelerator | | | | |

The components of Formulas A and B were packaged in the same composition prior to use, while the dicalcium phosphate dihydrate (DCPD) and tetracalcium phosphate (TTCP) were packaged in two separate compartments for Formula C with a 1:1 distribution of the contents of these compartments at the time of use to form the dental product of Formula C. The compositions in each of these two compartments are provided below.

TABLE 2

| Components | Mass percent |
|---|---|
| Water | 35.00% |
| DCPD | 35.00% |
| Kollidon 90F | 5.75% |
| BaSO4 | 20.00% |
| Aerosil ® 200 | 0.25% |
| Chlorhexidine | 1.00% |
| Propylene glycol | 3.00% |

TABLE 3

| Components | Mass percent |
|---|---|
| Propylene glycol | 32.25% |
| TTCP | 65.00% |
| Kollidon 90F | 0.75% |
| Aerosil ® 200 | 1.00% |
| Chlorhexidine | 1.00% |

The radiopaque contrast agent was barium sulfate in the case of Formulas A and C and zirconium oxide in the case of Formula B. The average particle size D50 of DCPD was 10 μm (9.213 μm measured by laser particle size analyzer) and the average particle size D50 of TTCP was less than 10 μm in each formula.

In moist conditions, Formula A produced a cement with a compressive strength of 3.27 MPa (the viscosity of Formula A was 98.2613 Pa·s), Formula B produced a cement with a compressive strength of 6.23 MPa (the viscosity of Formula B was 142.127 Pa·s) and Formula C produced a cement with a compressive strength of 4.34 MPa (the viscosity of Formula C was 93.2593 Pa·s).

The performance of the cements obtained in the context of the invention was evaluated in blind reworking tests on specimens and models of FKG teeth filled with different sealing materials.

Formula B above (B in photographs) as well as Formula A above (C in photographs) were tested in comparison with other commercially available sealants: “Total Fill BC Sealer” (A in photographs), “MTA Caps” (D in photographs) and “MTA Flow Extended Work Time” (“EWT” in photographs).

Prior to the degradation test, the setting of the sealing materials was checked by applying pressure to the top. All materials are hard and appear to be fully set. The degradation tests were performed using the Newtron P5XS ultrasonic generator at power 10 (maximum power of the endodontic range) and the ET25S insert, dedicated to the reworking of the coronal third and the isthmuses.

During the degradation tests on the specimens, the following observations were made: —the “Total Fill BC Sealer” cement is easily degraded using ultrasound, it even seems to be dissolved in contact with water (whitish color of the water after reworking);
the cements obtained from the formulas according to the invention are, like “Total Fill BC Sealer”, easily degraded using ultrasound. It also seems that dissolution is present but to a lesser extent than for “Total Fill BC Sealer”;
“the MTA Flow EWT” cement is easily degraded by ultrasound. However, it is not dissolved in contact with water;
the “MTA Caps” cement is more difficult to degrade than the other specimens and shows a higher strength. In addition, the sealing material darkens on prolonged contact with ultrasound.

When testing the degradation of the different sealing materials on the FKG tooth models, more visible differences became apparent. In fact, in deeper and narrower areas, the Total Fill BC Sealer has a higher strength than in the test specimen. It is possible to degrade the sealing material step by step because it is impossible to insert the insert deep into the canals. Some parts seem even more difficult to degrade and require a longer contact time with ultrasound. It is noted that with this sealing material, reworking is difficult in limited access such as narrow canals.

The cements obtained in the context of the invention have a reduced resistance compared with “Total Fill BC Sealer”. Indeed, when in contact with ultrasound, it is very easy to degrade the entire cement, even in limited access areas. It was also noted in this test that the formula containing zirconium oxide has a slightly higher resistance than the formula containing barium sulfate.

The reworking of root canal treatment using ultrasound is facilitated in the presence of the formulas according to the invention compared with existing sealing materials.

The expression “comprised between . . . and . . . ” should be understood to include the bounds.

1.-13. (canceled)

14. A dental product for forming an endodontic cement in a dental canal, said product comprising at least:
a powder composition in a mass content comprised between 40% and 55% comprising at least one calcium phosphate and able to form hydroxyapatite in the presence of moisture,
a binder in a mass content comprised between 0.5% and 5%, and
a liquid medium in a mass content comprised between 30% and 50%.

15. The product as claimed in claim 14, wherein the mass content of the powder composition is comprised between 43% and 52%.

16. The product as claimed in claim 14, wherein the powder composition comprises a mixture of dicalcium phosphate and tetracalcium phosphate.

17. The product as claimed in claim 14, wherein the powder composition has an average particle size D50 of less than or equal to 50 μm.

18. The product as claimed in claim 14, wherein the mass content of the binder is comprised between 0.5% and 3.5%.

19. The product as claimed in claim 14, further comprising a radiopaque contrast agent in a mass content of less than or equal to 15%.

20. The product as claimed in claim 14, further comprising a suspending agent in a mass content of less than or equal to 1.5%.

21. The product as claimed in claim 14, further comprising a cement setting accelerator in a mass content of less than or equal to 10%.

22. The product as claimed in claim 14, wherein:
the mass content of the powder composition is comprised between 43% and 52%,
the mass content of binder is comprised between 0.5% and 1%,
the liquid medium is non aqueous, and its mass content is comprised between 30% and 40%,
the product comprises a radiopaque contrast agent in a mass content comprised between 5% and 15%, and
the product comprises a cement setting accelerator in a mass content comprised between 4% and 7%.

23. The product as claimed in claim 22, wherein the radiopaque contrast agent comprises barium sulfate and wherein the mass content of the powder composition is comprised between 43% and 48%.

24. The product as claimed in claim 22, wherein the radiopaque contrast agent comprises zirconium oxide and wherein the mass content of the powder composition is comprised between 48% and 52%.

25. The product as claimed in claim 14, wherein:
the mass content of the powder composition is comprised between 43% and 52%,
the mass content of binder is comprised between 3% and 3.5%,
the liquid medium comprises water and its mass content is comprised between 30% and 40%, and
the product comprises a radiopaque contrast agent in a mass content comprised between 5% and 15%.

26. A kit for filling and sealing a dental canal, comprising at least the product as claimed in claim 14 in an introduction device suitable for introducing the product into the dental canal.
